(19) United States
(12) Patent Application Publication
(10) Pub. No.: US 2017/0080226 A1
(43) Pub. Date: Mar. 23, 2017

(54) SCREENING SYSTEM AND SCREENING METHOD FOR A COCHLEA IMPLANT DEVICE

(71) Applicant: ADVANCED BIONICS AG, Staefa (CH)

(72) Inventor: Idrick Akhoun, Hannover (DE)

(21) Appl. No.: 15/122,908

(22) PCT Filed: Mar. 17, 2014
(86) PCT No.: PCT/EP2014/055264

§ 371 Date: Aug. 31, 2016

(51) Int. Cl. A61N 1/36; A61N 1/372; A61B 5/12; A61N 1/05; A61B 5/00; A61B 5/04

(52) U.S. Cl. CPC A61N 1/36032; A61B 5/0031; A61B 5/04001; A61B 5/12; A61B 5/4851; A61B 5/6815; A61B 5/686; A61N 1/0541; A61N 1/37241

(57) ABSTRACT

A system includes a cochlear implant (CI) device and a screening device. The CI device includes an electrode array comprising a plurality of stimulation electrodes to be implanted in a patient's cochlea for electrical stimulation of the cochlea, a stimulation signal unit for generating stimulation signals to be supplied to the stimulation electrodes, and a unit for capturing ECAP signals induced at the electrodes in response to stimulation of the cochlea by applying stimulation signals to the electrodes. The screening device is adapted to cooperate with the stimulation signal unit in a manner so as to provide the electrodes with stimulation signals suitable for conducting growth function measurements, recovery measurements and spread-of-excitation measurements.

The invention relates to a screening system for a cochlea implant (CI) device and to a corresponding screening method.

Screening of a CI device during and after implantation is important in order to achieve an optimized performance of the CI device. Fast and reliable screening, which should also work when the patient is under anesthesia, is highly desirable in intraoperative settings in the operating theatre. A standard tool for evaluating the performance of an implanted CI device is the use of electrically-evoked compound action potentials (ECAP) which can be recorded on the intracochlear electrodes and sent back to the external processor of the CI device by back-telemetry. The ECAPs are recorded in response to electrical stimulation of the cochlea via at least one of the implanted electrodes in order to assess the basic functioning of the electrodes and integrity of the electrode-nerve interface. The ECAP is a voltage signal which comprises a negative and a smaller positive peak; the typical order of magnitude of the ECAP is between 1 and 1000 μV. To a first approximation, the ECAP magnitude is monotonically related to the amount of auditory nerve fibers that responded to the electrical stimulus.

The most important clinically relevant parameters which may be determined by screening processes include the neural response threshold (i.e. the minimal electrode current that generates a measurable ECAP, or the minimum interpulse interval at a given stimulation current, which generates a measurable ECAP) which may be determined from a linear fit of a so-called growth function measurement, the recovery time constant which may be obtained from an exponential fit of the recovery function (which is the ECAP at a given stimulation current as a function of the interpulse interval of the stimulation signal applied to the respective electrode), and the width of the electrical stimulation, as obtained from a fit of a spread-of-excitation (SOE) function (which corresponds to the ECAP of the electrodes adjacent to a probe electrode to which a stimulation signal is applied).

The article “AutoNRT: an automated system that measures ECAP thresholds with the Nucleus Freedom cochlea implant via machine intelligence” by A. Botros et al, in Artificial intelligence in Medicine (2007), 40, pages 15 to 28, relates to an automated ECAP threshold measurement algorithm, wherein there is automatic stimulation at low current levels around the smallest level to induce an ECAP and wherein it is detected whether or not a neural response is present in the recording, using a machine learning process. A similar description of such algorithm is also found in US 2011/0082521 A1 and US 2008/0319508 A1. In a retrospective evaluation study “Clinical Results of AutoNRT, a Completely Automatic ECAP Recording System for Cochlear Implants”, by B. van Dijk et al, Ear & Hearing (2007) 28(4), pages 558 to 570, it was found that in 90% of the cases the absolute difference between the result of the algorithm and the thresholds determined by a human observer was less than 9 current-levels, with the total timing of screening of 22 electrodes being estimated to 7 minutes.

US 2006/0287609 A1 and US 2007/0244410 A1 relate to methods of determining whether a neural response signal is a valid neural response signal or not, with artifacts being automatically removed from neural response signals, if necessary, wherein an artifact model is fitted to a neural recording signal, a strength-of-response metric is determined which describes a distance of the neural recording signal from the fitted artifact model signal, and the neural recording signal is identified as including a neural response signal if the strength-of-response metric is above a predetermined threshold.

US 2008/0221640 A1 and U.S. Pat. No. 7,206,640 B1 relate to a method for automatically determining the neural response threshold of each electrode from a linear fit of ECAP growth function measurements.

WO 03/070322 A1 relates to a method for neural response telemetry of a CI implant, wherein an automatic shape recognition algorithm is applied in order to classify automatically each response curve among a plurality of predetermined types of shapes in order to determine and optimize adjustment of the operation parameters of the CI device.

It is an object of the invention to provide for a screening system and screening method which is particularly user-friendly in the sense that it provides the clinically relevant information regarding the implanted CI device in a simple, reliable and efficient way.

According to the invention, this object is achieved by a system as defined in claims 1 and 15, respectively and methods as defined in claims 16 and 18, respectively.

The invention is beneficial in that, by accepting an ECAP measurement point as a valid ECAP-level only if the ECAP signal is at least 50 μV and the signal to noise ratio (SNR) is more than 5 dB, measurement that does not contain a true neural response can be identified in a simple and efficient manner, thereby avoiding to focus the analysis in a region-of-interest where it is impossible to record a response. This allows saving time and provides good-reliability results without the need of an expert clinician supervision in an overall reduced experimental time.

The aspect defined in claims 1 and 15 is particularly beneficial in that by combining an automated threshold current estimation with automated recovery measurements and automated SOE measurements, with the recovery measurements and/or the SOE measurements being conducted only for those electrodes for which an ECAP level of at least 100 μV was measured in the ECAP growth-function, all clinically relevant information can be obtained in a reliable and simple manner.

The aspect defined in claims 15 and 18 is particularly beneficial in that the growth function measurements can be conducted in a very efficient manner that avoids the current-unit range where no response is measurable.

Preferred embodiments of the invention are defined in the dependent claims.

Hereinafter, examples of the invention will be illustrated by reference to the attached drawings, wherein:

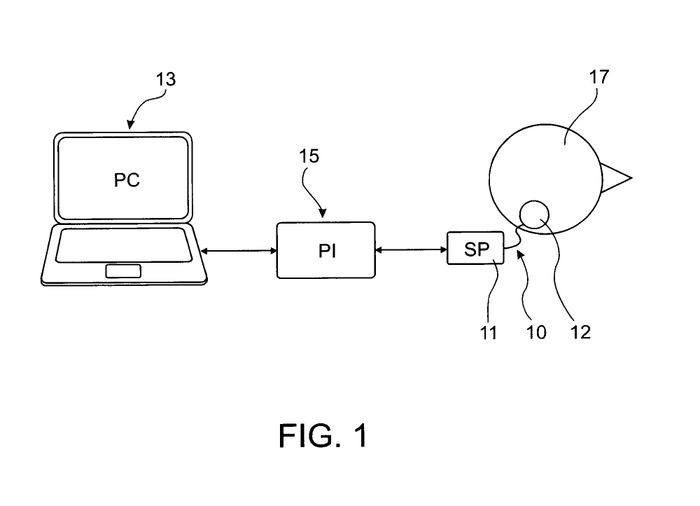

FIG. 1 is a schematic representation of an example of a screening system according to the invention;

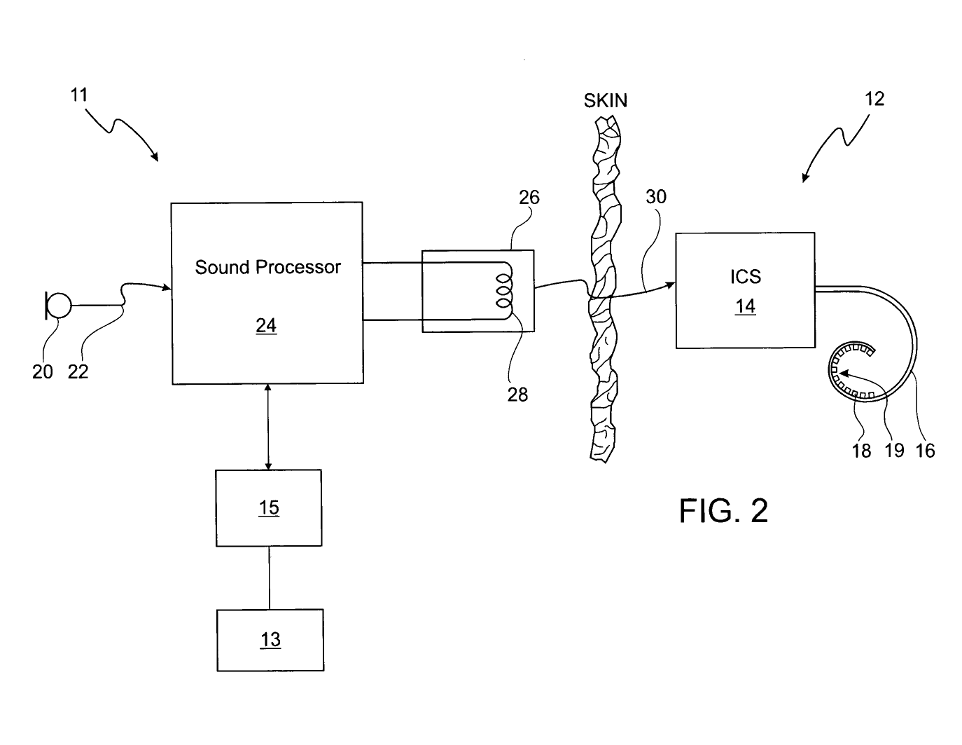

FIG. 2 is a schematic representation of an example of the CI device of the system of FIG. 1;

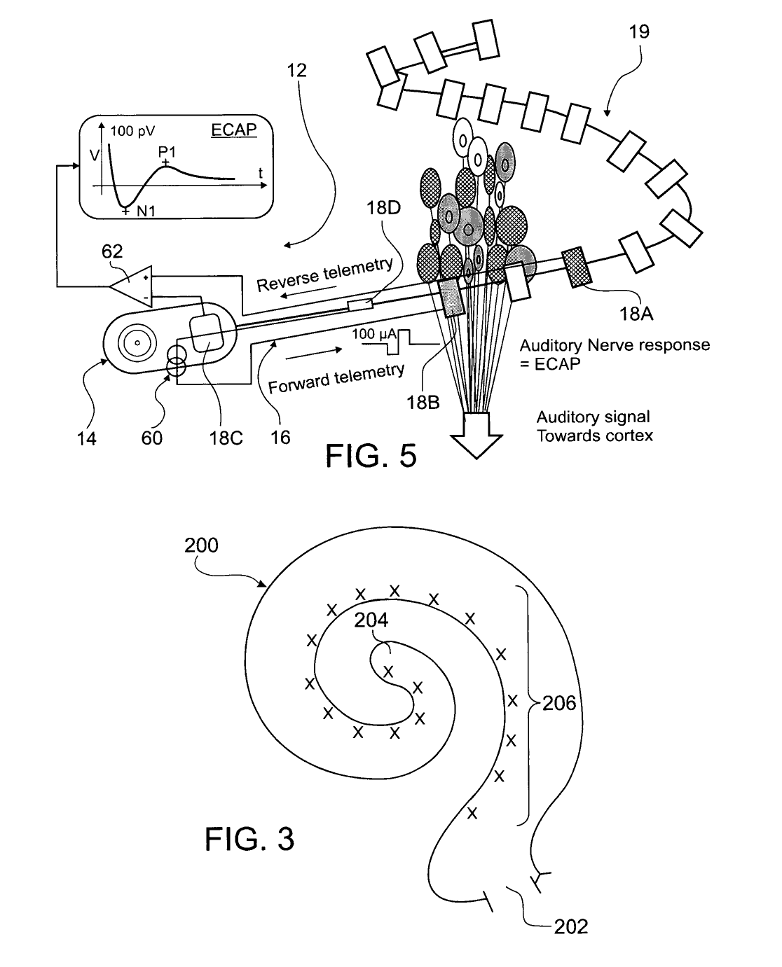

FIG. 3 is a schematic cross-sectional view of a human cochlea with marked stimulation sites;

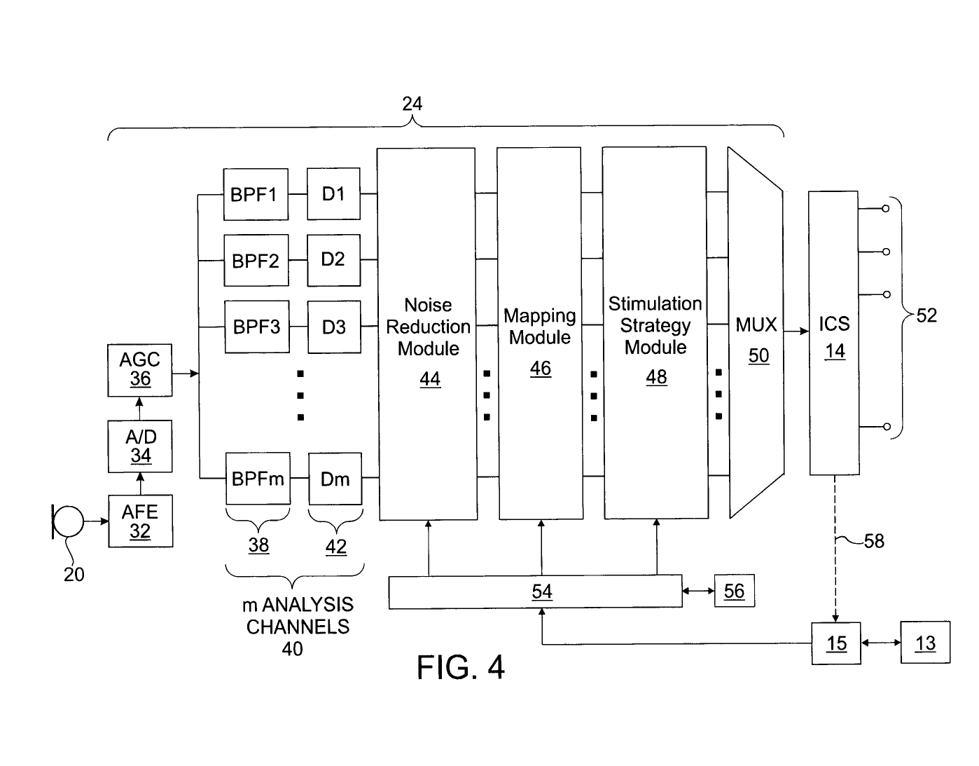

FIG. 4 is a block diagram of an example of the signal processing structure of a CI device to be used with the present invention;

FIG. 5 is a schematic illustration of a setup for ECAP measurements by reverse telemetry;

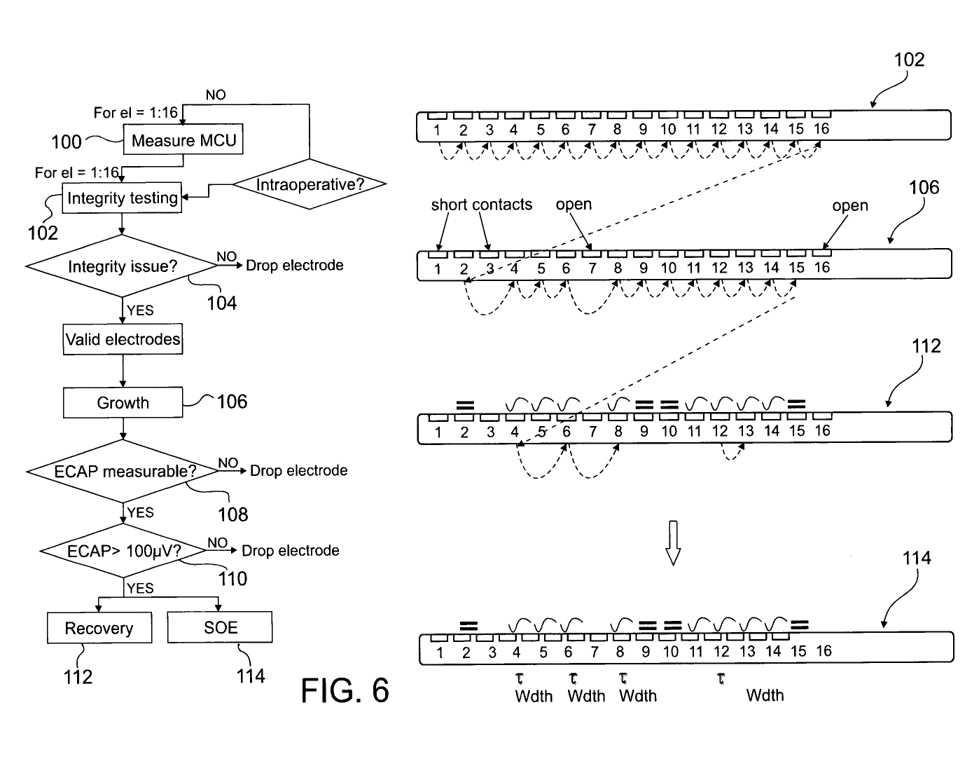

FIG. 6 is a flow diagram of an example of a screening process according to the invention;

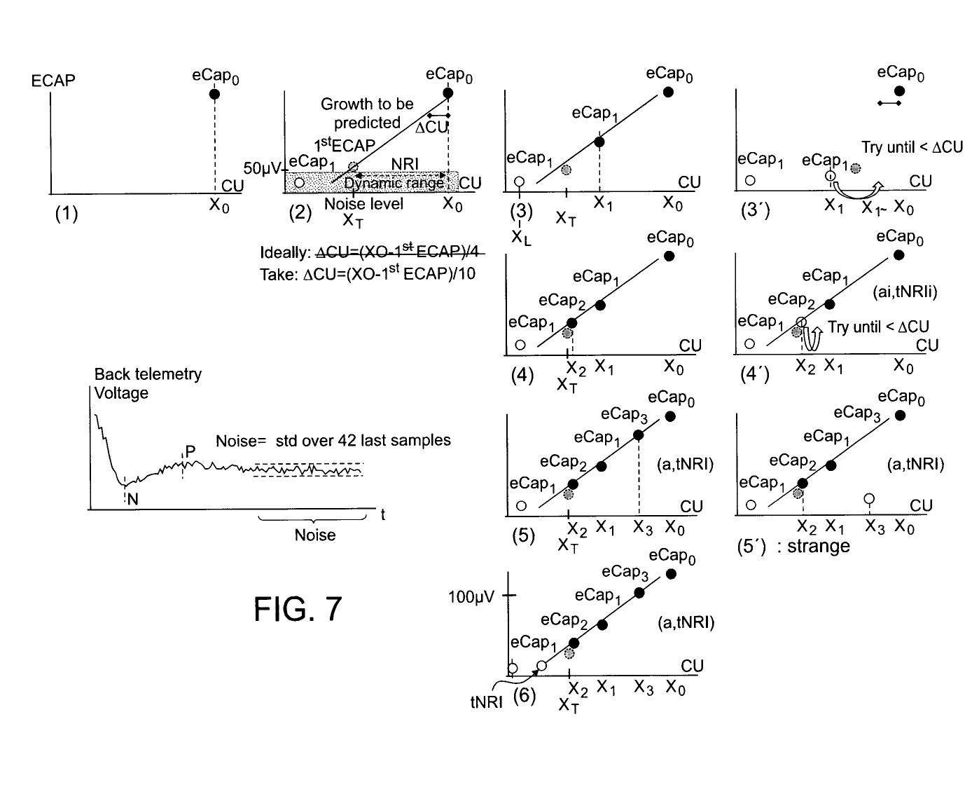

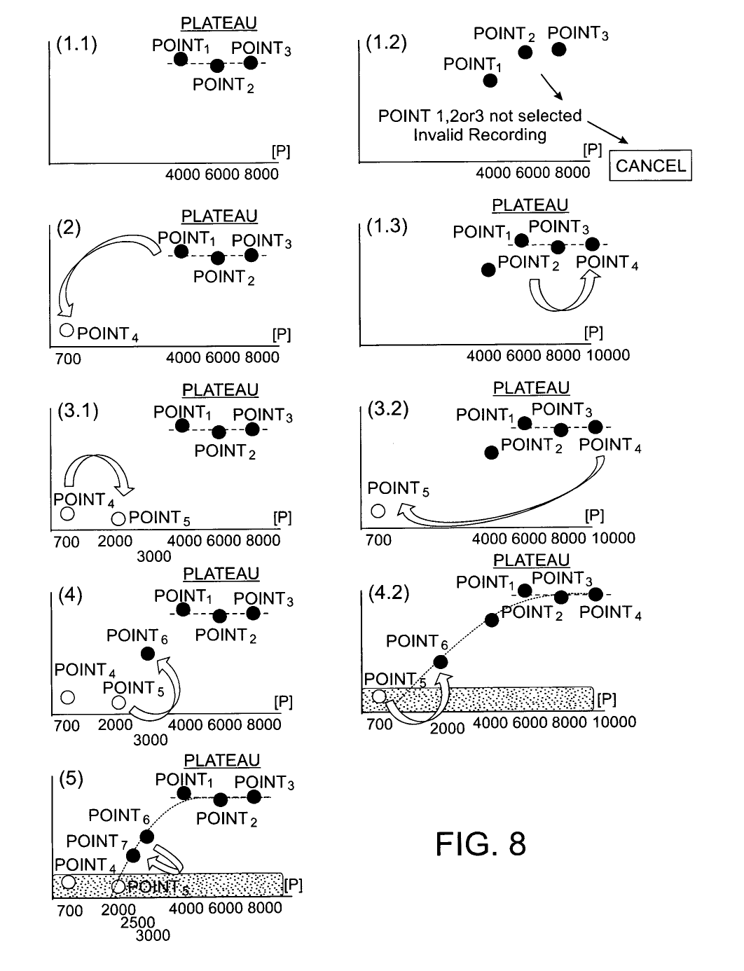

FIG. 7 is an illustration of an example of a growth function measurement procedure for determining the threshold electrode current to be used in the present invention, wherein a measurement sequence of several data points is shown;

FIG. 8 is an illustration of an example of a recovery measurement procedure to be used in the present invention, wherein a measurement sequence of several data points is shown; and

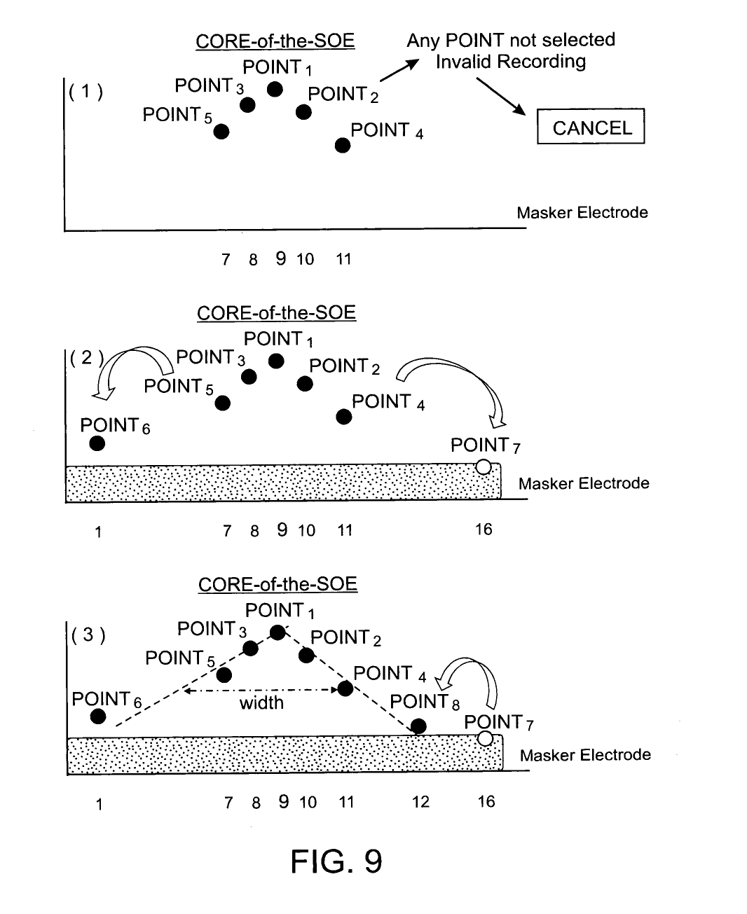

FIG. 9 is an illustration of an example of a SOE measurement procedure to be used in the present information, as shown, wherein a measurement sequence of several data points is shown.

FIG. 1 is a schematic representation of an example of a neural stimulation system according to the invention, comprising a screening device 13, which may be implemented as a computer, a programming interface 15 and a CI device 10 comprising a sound processing subsystem 11 and an implantable relation subsystem 12, with the CI device being worn by a patient 17. The screening device 13 communicates with the sound processing subsystem 11 via the programming interface 15, which may be implemented as a wired or wireless connection.

The screening device 13 serves to control the sound processing subsystem 11 such that test stimulation signals are applied to the patient 17 via the stimulation subsystem 12 and to evaluate or analyze the patient's neural response in the cochlea to the test stimulation signals, with the stimulation response being measured by the CI device 10 by recording the respective ECAP levels.

It is to be understood that the screening device 13 is used with the CI device 10 only for the screening procedure and, optionally, for adjustment/fitting of the CI device 10, but not during normal operation of the CI device 10.

In FIG. 2 an example of the cochlear implant device 10 of the system of FIG. 1 is shown schematically. The sound processing sub-system 11 serves to detect or sense an audio signal and divide the audio signal into a plurality of analysis channels, each containing a frequency domain signal (or simply “signal”) representative of a distinct frequency portion of the audio signal. A signal level value and a noise level value are determined for each analysis channel by analyzing the respective frequency domain signal, and a noise reduction gain parameter is determined for each analysis channel as a function of the signal level value and the noise level value of the respective analysis channel. Noise reduction is applied to the frequency domain signal according to the noise reduction gain parameters to generate a noise reduced frequency domain signal. Stimulation parameters are generated based on the noise reduced frequency domain signal and are transmitted to the stimulation sub-system 12.

Stimulation sub-system 12 serves to generate and apply electrical stimulation (also referred to herein as “stimulation current” and/or “stimulation pulses”) to stimulation sites at the auditory nerve within the cochlear of a patient 17 in accordance with the stimulation parameters received from the sound processing sub-system 11. Electrical stimulation is provided to the patient 17 via a CI stimulation assembly 18 comprising a plurality of stimulation channels, wherein various known stimulation strategies, such as current steering stimulation or N-of-M stimulation, may be utilized. In addition, the stimulation assembly 18 also is used for ECAP measurements via reverse telemetry, as will be described in more detail with regard to FIG. 5 below.

The stimulation parameters may control various parameters of the electrical stimulation applied to a stimulation site including, but not limited to, frequency, pulse width, amplitude, waveform (e.g., square or sinusoidal), electrode polarity (i.e., anode-cathode assignment), location (i.e., which electrode pair or electrode group receives the stimulation current), burst pattern (e.g., burst on time and burst off time), duty cycle or burst repeat interval, spectral tilt, ramp on time, and ramp off time of the stimulation current that is applied to the stimulation site.

FIG. 3 illustrates a schematic structure of the human cochlea 200. As shown in FIG. 3, the cochlea 200 is in the shape of a spiral beginning at a base 202 and ending at an apex 204. Within the cochlea 200 resides auditory nerve tissue 206 which is organized within the cochlea 200 in a tonotopic manner. Low frequencies are encoded at the apex 204 of the cochlea 200 while high frequencies are encoded at the base 202. Hence, each location along the length of the cochlea 200 corresponds to a different perceived frequency. Stimulation subsystem 12 is configured to apply stimulation to different locations within the cochlea 200 (e.g., different locations along the auditory nerve tissue 206) to provide a sensation of hearing.

Returning to FIG. 2, sound processing subsystem 11 and stimulation subsystem 12 is configured to operate in accordance with one or more control parameters. These control parameters may be configured to specify one or more stimulation parameters, operating parameters, and/or any other parameter as may serve a particular application. Exemplary control parameters include, but are not limited to, most comfortable current levels (“M levels”), threshold current levels (“T levels”), dynamic range parameters, channel acoustic gain parameters, front and backend dynamic range parameters, current steering parameters, amplitude values, pulse rate values, pulse width values, polarity values, filter characteristics, and/or any other control parameter as may serve a particular application.

In the example shown in FIG. 2, the stimulation sub-system 12 comprises an implantable cochlear stimulator (“ICS”) 14, a lead 16 and the stimulation assembly 18 disposed on the lead 16. The stimulation assembly 18 comprises a plurality of “stimulation contacts” 19 for electrical stimulation of the auditory nerve. The lead 16 may be inserted within a duct of the cochlea in such a manner that the stimulation contacts 19 are in communication with one or more stimulation sites within the cochlea, i.e. the stimulation contacts 19 are adjacent to, in the general vicinity of, in close proximity to, directly next to, or directly on the respective stimulation site.

In the example shown in FIG. 2, the sound processing sub-system 11 is designed as being located external to the patient 17; however, in alternative examples, at least one of the components of the sub-system 10 may be implantable.

In the example shown in FIG. 2, the sound processing sub-system 11 comprises a microphone 20 which captures audio signals from ambient sound, a microphone link 22, a sound processor 24 which receives audio signals from the microphone 20 via the link 22, and a headpiece 26 having a coil 28 disposed therein. The sound processor 24 is configured to process the captured audio signals in accordance with a selected sound processing strategy to generate appropriate stimulation parameters for controlling the ICS 14 and may include, or be implemented within, a behind-the-ear (BTE) unit or a portable speech processor (“PSP”). In the example of FIG. 2 the sound processor 24 is configured to transcutaneously transmit data (in particular data representative of one or more stimulation parameters) to the ICS 14 via a wireless transcutaneous communication link 30. The headpiece 26 may be affixed to the patient's head and positioned such that the coil 28 is communicatively coupled to the corresponding coil (not shown) included within the ICS 14 in order to establish the link 30. The link 30 may include a bidirectional communication link and/or one or more dedicated unidirectional communication links. According to an alternative embodiment, the sound processor 24 and the ICS 14 may be directly connected by wires.

In FIG. 4 a schematic example of a sound processor 24 is shown. The audio signals captured by the microphone 20 are amplified in an audio front end circuitry 32, with the amplified audio signal being converted to a digital signal by an analog-to-digital converter 34. The resulting digital signal is then subjected to automatic gain control using a suitable automatic gain control (AGC) unit 36.

After appropriate automatic gain control, the digital signal is subjected to a filterbank 38 comprising a plurality of filters F1 . . . Fm (for example, band-pass filters) which are configured to divide the digital signal into m analysis channels 40, each containing a signal representative of a distinct frequency portion of the audio signal sensed by the microphone 20. For example, such frequency filtering may be implemented by applying a Discrete Fourier Transform to the audio signal and then divide the resulting frequency bins into the analysis channels 40.

The signals within each analysis channel 40 are input into an envelope detector 42 in order to determine the amount of energy contained within each of the signals within the analysis channels 40 and to estimate the noise within each channel. After envelope detection the signals within the analysis channels 40 are input into a noise reduction module 44, wherein the signals are treated in a manner so as to reduce noise in the signal in order to enhance, for example, the intelligibility of speech by the patient. Examples of the noise reduction module 44 are described in WO 2011/032021 A1.

The noise reduced signals are supplied to a mapping module 46 which serves to map the signals in the analysis channels 40 to the stimulation channels S1 . . . Sn. For example, signal levels of the noise reduced signals may be mapped to amplitude values used to define the electrical stimulation pulses that are applied to the patient 17 by the ICS 14 via M stimulation channels 52. For example, each of the m stimulation channels 52 may be associated to one of the stimulation contacts 19 or to a group of the stimulation contacts 19.

The sound processor 24 further comprises a stimulation strategy module 48 which serves to generate one or more stimulation parameters based on the noise reduced signals and in accordance with a certain stimulation strategy (which may be selected from a plurality of stimulation strategies). For example, stimulation strategy module 48 may generate stimulation parameters which direct the ICS 14 to generate and concurrently apply weighted stimulation current via a plurality 52 of the stimulation channels S1 . . . Sn in order to effectuate a current steering stimulation strategy. Additionally or alternatively the stimulation strategy module 48 may be configured to generate stimulation parameters which direct the ICS 14 to apply electrical stimulation via only a subset N of the stimulation channels 52 in order to effectuate an N-of-M stimulation strategy.

The sound processor 24 also comprises a multiplexer 50 which serves to serialize the stimulation parameters generated by the stimulation strategy module 48 so that they can be transmitted to the ICS 14 via the communication link 30, i.e. via the coil 28.

The sound processor 24 may operate in accordance with at least one control parameter which is set by a control unit 54. Such control parameters may be the most comfortable listening current levels (MCL), also referred to as “M levels”, threshold current levels (also referred to as “T levels”), dynamic range parameters, channel acoustic gain parameters, front and back end dynamic range parameters, current steering parameters, amplitude values, pulse rate values, pulse width values, polarity values and/or filter characteristics. Examples of such auditory prosthesis devices, as described so far, can be found, for example, in WO 2011/032021 A1.

The stimulation strategy module 48 also controls the shape of the stimulation pulses. In general, the pulse shape is determined by a shape parameter set including at least one shape parameter. Such shape parameter set may be stored in a memory 56.

In addition, the stimulation response to test pulses applied via the electrodes 18 of the electrode array 19 is measured, and the respective stimulation response data is supplied to the screening device 13 for evaluating the stimulation response. The stimulation response data is obtained from ECAPs measurements carried out by the CI device 10, with the evoked potential data being recorded by reverse telemetry from the ICS 14 to the sound processing subsystem 11, from where the data is supplied via the programming interface 15 to the screening device 13 (this path is schematically indicated at 58 in FIG. 4).

FIG. 5 shows a schematic illustration of an example of auditory nerve excitation and the resulting ECAP recording at electrode 18A by reverse telemetry, following electrical stimulation at electrode 18B by forward telemetry (the stimulated neurons are indicated by dark grey circles in FIG. 8, the return electrodes are indicated at 18C and 18D, respectively). The current-source 60 and the amplifiers 62 are positioned inside the receiver part of the ICS 14. The typical ECAP peaks are indicated at N1 and P1 in example of an ECAP signal vs. time in FIG. 5. The peaks may be used as markers to measure the ECAP amplitude as the differential voltage between P1 and N1.

An example of ECAPs measurements which are used for deriving tNRI (neural response imaging) levels is described in “Comparisons between neural response imaging thresholds, electrically evoked auditory reflex thresholds and most comfortable loudness levels in CII bionic ear users with HiResolution sound processing strategies”, by D. M. Han et al., in Acta Otolaryngol 125(7), 2005, p. 732-735.

The screening device 13 is adapted to automatically conduct a screening process for evaluating the electrode array 19 in the implanted state in order to determine those of the electrodes 18 which function properly, to estimate the hearing perception threshold current for each properly functioning electrode, to obtain an estimation of the recovery time constant from an exponential fit of a recovery function and the width of electrical stimulation from a fit of a SOE function for each of the properly functioning electrodes which have proved in the growth function measurements to have a large-enough ECAP level of more than 100 μV.

FIG. 6 shows a block diagram of an example of a screening process to be conducted by the screening device 13. In a first step 100 the maximum tolerable current level (MCU) may be determined for each electrode in order to prevent over-stimulating the patient. Since such determination requires feedback of the patient, it is not possible to determine the MCU in intra-operative settings where the patient is under anesthesia; in such cases a default value of the maximum allowable current may be used rather than empirically determining the MCU. Thus, the MCU measurement of step 100 is an optional non-automated step which requires feedback by the patient.

In a second step 102, the integrity of each electrode is checked, typically by measuring the impedance of each electrode, wherein it is judged that an electrode is a valid electrode if the measured impedance is within a given range (if the impedance is too high, this is an indication that the contact is open, and if the impedance is too low, this is an indication that the contact is short). Any invalid electrode is discarded both for stimulation and ECAP recording. The integrity judgment step is indicated at 104 in FIG. 6.

In step 106 a smart growth measurement is performed for each valid electrode in order to obtain the ECAP threshold current from a linear fit of the obtained measurement points. In addition, the smart growth measurement procedure identifies those of valid electrodes for which an ECAP is at all measurable and those electrodes for which the measurable ECAP is more than 100 μV (see boxes 108 and 110 in FIG. 6), with only electrodes fulfilling these two conditions being later used for conducting a recovery function measurement (step 112) for determining the recovery time constant for these electrodes and in a SOE measurement (step 114) for determining the width of electrical stimulation for these electrodes.

In FIG. 7 an example of the growth function measurement procedure is illustrated by several diagrams showing the measured ECAP as a function of the stimulation current CU, wherein diagram (1) corresponds to the first measurement step and diagram (6) corresponds to the last measurement step.

Generally, for each ECAP measurement, a neural response image (NRI) is measured by recording three or four buffers (depending on the artifact rejection technique chosen), and it is automatically checked for anomalies, such as abnormal noise or NRI amplifier distortion. In case that no anomalies are found, the ECAP level is determined from the first part of the NRI signal (back-telemetry voltage as a function of time) by determining the differential voltage between the signal minimum (indicated at “N” in diagram (0) of FIG. 7) and the maximum (indicated at “P” in diagram (0) of FIG. 7); this voltage difference is a measure of the ECAP level for the respective stimulation signal.

Any recording is judged to be a valid recording only in case that the ECAP level is at least 50 μV and the signal to noise ratio (SNR) is at least +5 dB, with the signal value being taken as the RMS (root mean square) or the first portion of the NRI signal and the noise value being calculated as the RMS over the signal portion towards the end of the measurement, where the time averaged voltage level is substantially constant (see signal part labeled “noise” in FIG. 7).

In a first step, as shown in diagram (1) of FIG. 7, a first ECAP level is determined at, or at at least 70% of, the maximum allowable electrode current X₀. In case that no valid/acceptable ECAP level can be determined, the recording parameters may be adapted, and if still no valid ECAP level is obtained, the growth function measurement at that electrode is aborted in favor of a growth function measurement at the next valid electrode.

In a second step, shown in diagram (2) of FIG. 7, an ECAP measurement is conducted at a second current level X_{L} which is so low that no valid ECAP level is expected (indicated at “ECAP1” in diagram (2), i.e. at a current level which is below the (roughly) expected ECAP threshold current level (indicated at “first ECAP” in diagram (2)); in diagram (2) the noise level range is from 0 to 50 μV (ECAP levels within this range are considered to be invalid), and the dynamic range of the electrode extends is considered to extend from the current level corresponding to the “first ECAP level” to the maximum allowable current X₀.

Further, a minimum distance ΔCU is defined, which may be, for example, 10% of the difference between the maximum allowable current and the lowest current tested (X_{L}), with only measurements being accepted having a (current) distance which is at least the minimum distance ΔCU.

Preferably, the low current level X_{L} used in step (2) is not more than 50% of the maximum allowable current level X₀.

In a third step, as illustrated in diagram (3) of FIG. 7, an ECAP measurement is conducted at a third current level X₁ in between the low current level X_{L} and the maximum current level X₀. In case that at the selected third current level no valid ECAP level is obtained, the value of the third current level may be increased in a stepwise manner until a valid ECAP level is measured; however, the finally selected third current level has to be spaced apart from the maximum current level X₀ by at least ΔCU (see diagram (3′)).

In a forth step, as illustrated in diagram (4) of FIG. 7, an ECAP level (indicated at “ECAP2” in diagram (4)) is measured a forth current level X₂ which is close to (within +1-25%) of the current level X_{T} corresponding to the estimated ECAP threshold current level; the threshold current level X_{T} may be estimated from the slope and position of the measurement points obtained so far. If no valid ECAP level is obtained, the value of the fourth current level X₂ may be increased in a stepwise manner until a valid ECAP level is measured (as illustrated in diagram (4′); however, the distance to the measurement point at the current level X₁ has to be at least ΔCU).

In a fifth step, as illustrated in diagram (5), a further ECAP measurement is conducted at a fifth current level X₃ in between the first current level X₀ and the third current level X₁.

The growth measurement is successfully completed as soon as at least four valid points have been obtained on one electrode. In case that in one of the current level selection steps necessary for obtaining a valid ECAP level it is not possible to fulfill the requirement that there is at least the minimum distance ΔCU to current levels already used in an ECAP measurement, the growth function measurement at the respective electrode is aborted.

The procedure therefore continues measuring points until either four valid points are obtained or measurement is aborted. Then, ECAP thresholds ECAP1 is estimated from a linear fit to the four valid measurement points. This is illustrated in diagram (6) as the sixth step.

Once the function measurement at one electrode is terminated, the screening device 13 continues with a growth function measurement of a next electrode.

After the growth function measurements have been completed for all valid electrodes, a recovery measurement procedure is optionally applied to all valid electrodes which have shown in the growth function measurements an ECAP level of at least 100 μV.

The growth function measurement procedure for one electrode is illustrated in FIG. 8, including various diagrams wherein the ECAP value is shown as a function of the interpulse interval (IPI). In such recovery measurements, the stimulation current is kept constant, while the IPI is varied from one measurement point to the next measurement point.

In a first step, which is illustrated in diagram (1.1) of FIG. 8, ECAP measurements are conducted in a plateau region at at least a first IPI, such as 4000 μs, at a second IN higher than the first IPI, such as 6000 μs, and a third IPI higher than the second IPI, such as 8000 μs, with the IPI values being selected such that the measured ECAP levels are almost similar do not differ for example by more than 20%. As in the case of the growth function measurements, each measurement is checked for anomalies, and an ECAP measurement is accepted only if the level is at least 50 μV and the SNR is at least +5 dB. Buffers are measured and are possibly reused, at least some of them, within the same electrode (in the same sweep).

The first, second and third IPI may be changed in case that the resulting ECAP levels differ too much. For example, if the ECAP level measured at 4000 μs is found to be too low, the ECAP measurement may be conducted at 10000 μs (as illustrated in diagram 1.3) of FIG. 8).

If it is not possible to obtain three plateau measurement points, the recovery measurement procedure at the respective electrode is aborted, and a recovery measurement procedure is applied to the next valid electrode (see diagram (1.2) of FIG. 8).

In a second step, as illustrated in diagram (2) of FIG. 8, a ECAP measurement is conducted at a fourth IPI value which is relatively small (typically not more than 20% of the first IPI), such as 700 μs.

In a third step, as illustrated in diagram (3.1) of FIG. 8, an ECAP measurement at a fifth IPI between the fourth IPI and the first IPI is conducted in case that no valid ECAP level was obtained at the fourth IPI (in the example of diagram (3.1), the fifth IPI is 2000 μs). In case that still no valid ECAP level is obtained, the fifth IPI may be increased in a stepwise manner until a valid ECAP level is measured (in the example shown diagram (4) of FIG. 8 the increased IPI is 3000 μs, where a valid ECAP level is obtained). In case that a valid ECAP level is obtained at the fourth IPI, the fifth IPI would be below the fourth IPI.

In a fourth step, which is illustrated in diagram (5) of FIG. 8, at least one further ECAP measurement is conducted at a further IPI which is between the fifth IPI and the first IPI (in the example of diagram (5) at 2500 μs).

Preferably, the measurement procedure is continued with additional ECAP measurements at further IPIs until seven valid measurement points altogether are obtained.

In a fifth step, an exponential fit is applied to the valid ECAP measurements of the previous steps in order to obtain the recovery time constant.

While the left-hand diagrams of FIG. 8 show an example with a relatively high minimum IPI being necessary to obtain a valid ECAP level, in the right-hand column of FIG. 8 an example is illustrated in which the minimum IPI necessary for obtaining a valid ECAP level is relatively low.

After recovery measurements have been completed on all valid electrodes which have shown in the growth function measurements an ECAP level of at least 100 μV, spread-of-excitation is optionally conducted on these electrodes.

In FIG. 9 an example of a SOE measurement procedure is illustrated by diagrams showing the measured ECAP level as a function the respective electrode, with electrode number 9 being stimulated as the probe electrode.

In SOE measurements, one of the valid electrodes having an ECAP level of at least 100 μV as measured in the function measurements is selected as the probe electrode and is stimulated by a stimulation signal having a stimulation current in the dynamic range between the current level resulting in a ECAP level of 100 μV and the maximum allowable electrode current, with the resulting ECAP level being measured in the adjacent electrodes in order evaluate the width of the electrical stimulation (in general, stimulation at one of the electrodes always results in some stimulation at the adjacent electrodes).

In the example of FIG. 9, electrode number 9 is selected as the probe electrode, and in a first step the resulting ECAP levels induced at the probe electrode itself, at the two closest electrodes in both directions (electrodes numbers 8 and 10 in the example of FIG. 9 and at the two second closest electrodes in both directions from the probe electrode (in the example of FIG. 9 electrodes 7 and 11) are measured subsequently (these five electrodes are considered to represent the “SOE-core” electrodes). An example of the ECAP levels obtained at the “core electrodes” is illustrated in diagram (1) of FIG. 9.

As in the above discussed growth function and recovery measurements, also in the SOE measurements an ECAP level is accepted only if the amplitude is more than 50 μV and the SNRs higher than 5 dB. In case that at least one of the “core electrodes” does not produce a valid ECAP level, the SOE measurement procedure at the selected probe electrode is aborted.

In a second step, as illustrated in diagram (2) of FIG. 9, the ECAP levels are measured at the most apical electrode (electrode number 1) and the most basal electrode (electrode number 16), while the probe electrode number 9 is stimulated (in the example of diagram (2), no valid ECAP level is obtained at electrode number 16).

In a third step, as illustrated in diagram (3) of FIG. 9, additional electrodes between the most extreme electrodes number 11 and 16 and the “core electrodes” may be measured (in the example of diagram (3) a valid ECAP level is obtained for electrode number 12). Such measurement of additional electrodes (while the probe electrode is stimulated) may be continued until for at least a third (or half) of the electrodes a valid ECAP response has been obtained on each side. For example, if at least half of the electrodes in each side of the probe electrode should have a valid ECAP response, assuming that the probe electrode is electrode number 11, then among electrodes 1 to 10 at least five valid ECAP levels should have been obtained, and at least three valid ECAP levels should have been obtained for electrodes numbers 12 to 16.

Once the SOE measurements have been completed, the obtained data points are fitted with an appropriate function in order to obtain the width of electrical stimulation of the respective probe electrode as an output of the SOE measurement procedure (see diagram (3) of FIG. 9).

1. A system comprising a cochlear implant device and a screening device communicating with the cochlear implant device,
the cochlear implant device comprising:
an electrode array comprising a plurality of stimulation electrodes to be implanted in a patient's cochlea for electrical stimulation of the cochlea,
a stimulation signal unit for generating stimulation signals to be supplied to the stimulation electrodes,
a unit for capturing ECAP signals induced at the electrodes in response to stimulation of the cochlea by applying stimulation signals to the electrodes;
the screening device being adapted to cooperate with the stimulation signal unit in a manner so as to provide the electrodes with stimulation signals suitable for conducting growth function measurements, recovery measurements and spread-of-excitation measurements, and being adapted to
estimate, for at least part of the electrodes, the threshold current level required for achieving an ECAP-signal based on interpolated growth function measurements of the ECAP-signal at at least four different current levels;
conduct recovery measurements for at least some of those electrodes for which an ECAP level of at least 100 μV was measured in the growth function measurements, the recovery measurements comprising ECAP measurements at constant current level as a function of an interpulse interval of the stimulation signal in order to obtain a recovery function for each of the recovery-measured electrodes, and obtain a recovery time constant for each of the recovery-measured electrodes from an exponential fit of the respective recovery function; and
conduct a spread-of-excitation (SOE) measurement for at least some of those electrodes for which an ECAP level of at least 100 μV was measured in the growth function measurements, wherein the stimulation current is applied subsequently only to one of the electrodes as the probe electrode while an ECAP level is measured subsequently at each of the valid electrodes in order to obtain a spread-of-excitation function; and obtain, for each electrode used as a probe electrodes a width of electrical stimulation from a fit of the spread-of-excitation function;
wherein a measurement point in all measurements is accepted by the screening device as a valid ECAP measurement only if the ECAP signal is at least 50 μV and the signal-to-noise ratio is more than 5 dB, with the noise level being measured at times when there is no electrical stimulation of the cochlea.

2. The system of claim 1, wherein the noise level of the ECAP-measurement is calculated as an RMS value from the levels measured towards the end of the ECAP-measurement, where a time-averaged level is substantially constant.

3. The system of claim 2, wherein the signal level of the ECAP-measurement is calculated as an RMS value from the levels of a first portion of the ECAP-measurement.

4. The system of claim 1, wherein the screening device is adapted to cooperate with the cochlear implant device in a manner to measure, prior to conducting the growth function measurements, the impedance of each electrode, and to judge that an electrode is a valid electrode which is to used be in the growth function measurements if the measured impedance is within a given range.

5. The system of claim 1, wherein the screening device is adapted to conduct the growth function measurement in a manner that it comprises the following steps:
a first step with an ECAP measurement at a first current level, which is at least 70% of the maximum allowable current level;
a second step with an ECAP measurement at a second current level which is not more than 50% of the maximum allowable current level, with no valid ECAP-level being expected at the second current level;
a third step with an ECAP measurement at a third current level in-between the first current level and the second current level, wherein, if no valid ECAP-level can be measured at the third current level, the value of the third current level is increased in a stepwise manner until a valid ECAP-level is measured;
a fourth step, wherein an ECAP threshold is estimated from the slope of the valid ECAP levels obtained in the first to third steps, and an ECAP-level is measured at a fourth current level close to, within +/−25% of, the current level corresponding to the estimated ECAP-threshold, and wherein, if no valid ECAP-level can be measured at the fourth current level, the value of the fourth current level is increased in a stepwise manner until a valid ECAP-level is measured; and
a fifth step with an ECAP measurement at a fifth current level in-between the first current level and the third current level, wherein the fifth current level, if necessary, may be increased in a stepwise manner until a valid ECAP-level is measured;
wherein the ECAP threshold is estimated from the slope of a linear fit of the valid ECAP levels obtained in the previous steps.

6. The system of claim 5, wherein if no valid ECAP is obtained in first step, the growth function measurement at that electrode is aborted.

7. The system of claim 5, wherein the growth function measurement at that electrode is aborted if the next current level to be applied is has a distance to an already measured current level which is less than a given minimum distance.

8. The system of claim 7, wherein the minimum distance is set as not more than 10% of the difference between the maximum allowable current level and the lowest current tested.

9. The system of claim 1, wherein the screening device is adapted to conduct the recovery measurement in a manner that comprises the following steps:
a first step with ECAP measurements in a plateau region at at least a first interpulse interval of the stimulation signal, a second interpulse interval higher than the first interpulse interval and a third interpulse interval higher than the second interpulse interval, with the first, second and third interpulse intervals being selected such that the measured ECAP levels do not differ by more than 20%;
a second step with an ECAP measurement at a fourth interpulse interval which is not more than 20% of the third interpulse interval,
a third step with an ECAP measurement at a fifth interpulse interval which is between the fourth interpulse interval and the first interpulse interval if no valid ECAP level was obtained at the fourth interpulse interval, with value the fifth interpulse interval being increased in a stepwise manner until a valid ECAP-level is measured, and which is below the fourth interpulse interval if a valid ECAP level was obtained at the fourth interpulse interval,
a fourth step with ECAP measurements at at least one further interpulse interval which is between the fifth interpulse interval and the first interpulse interval,
a fifth step wherein an exponential fit is applied to the valid ECAP measurements of the previous steps in order to obtain the recovery time constant.

10. The system of claim 1, wherein the screening device is adapted to conduct the SOE measurement in a manner that it comprises the following steps:
a first step wherein a stimulation signal is applied to one of the electrodes selected as a probe electrode while ECAP-levels are measured for the SOE-core electrodes which consist of the probe electrode and the closest and second-closest adjacent electrodes in both directions with regard to the probe electrode, and wherein the measurement of that probe electrode is aborted if for at least one of the SOE-core electrodes no valid ECAP-level is obtained;
a second step wherein the stimulation signal of the first step is applied to the probe electrode while ECAP-levels are measured at the most apical and the most basal electrode of the electrode array, respectively;
a third step wherein the stimulation signal of the first step is applied to the probe electrode while ECAP-levels are measured at electrodes between the core electrodes and the most apical and the most basal electrode until a valid ECAP-level is obtained for at least one third of the electrodes at the apical side of the probe electrode and at the basal side of the probe electrode, respectively; and
a fourth step wherein a fit is applied to the valid ECAP measurements of the previous steps in order to obtain the width of electrical stimulation for the probe electrode.

11. The system of claim 9, wherein the screening device is adapted to determine for each valid electrode the current resulting in an ECAP level of 100 μV, and wherein the control unit is adapted to apply in the recovery measurements and/or in the SOE measurements the current for each electrode such that it is at least the determined current resulting in an ECAP level of 100 μV but not more than a set maximum current.

12. The system of claim 1, wherein the cochlear implant device comprises means for providing an input audio signal and a sound processor for generating a neural stimulation signal from the input audio signal, with the sound processor comprising the stimulation signal unit.

13. The system of claim 12, wherein the unit for capturing ECAP signals comprises the stimulation electrodes and is adapted to transmit the response data via a reverse telemetry link to the sound processor.

14. The system of claim 1, wherein the screening device is implemented by a computer communicating with the cochlear implant device via a programming interface.

15. A system comprising a cochlear implant device and a screening device communicating with the cochlear implant device,
the cochlear implant device comprising:
an electrode array comprising a plurality of stimulation electrodes to be implanted in a patient's cochlea for electrical stimulation of the cochlea,
a stimulation signal unit for generating stimulation signals to be supplied to the stimulation electrodes,
a unit for capturing ECAP signals induced at the electrodes in response to stimulation of the cochlea by applying stimulation signals to the electrodes;
the screening device being adapted to cooperate with the stimulation signal unit in a manner so as to provide the electrodes with stimulation signals suitable for conducting growth function measurements, and being adapted to conduct a growth function measurement for estimating the threshold current level required for achieving a valid ECAP-signal at a certain electrode, the growth function measurement comprising the following steps:
a first step with an ECAP measurement at a first current level, which is at least 70% of the maximum allowable current level;
a second step with an ECAP measurement at a second current level which is not more than 20% of the maximum allowable current level, with no valid ECAP-level being expected at the second current level;
a third step with an ECAP measurement at a third current level in-between the first current level and the second current level, wherein, if no valid ECAP-level can be measured at the third current level, the value of the third current level is increased in a stepwise manner until a valid ECAP-level is measured;
a fourth step, wherein an ECAP threshold is estimated from the slope of the valid ECAP levels obtained in the first to third steps, and an ECAP-level is measured at a fourth current level close to, within +/−25% of, the current level corresponding to the estimated ECAP threshold, and wherein, if no valid ECAP-level can be measured at the fourth current level, the value of the fourth current level is increased in a stepwise manner until a valid ECAP-level is measured;
a fifth step with an ECAP measurement at a fifth current level in-between the first current level and the third current level, wherein the fifth current level, if necessary, may be increased in a stepwise manner until a valid ECAP-level is measured;
wherein the ECAP threshold is estimated from the slope of a linear fit of the valid ECAP levels obtained in the previous steps.
wherein a measurement point in all measurements generally is accepted by the analysis unit as a valid ECAP measurement only if the ECAP signal is at least 50 μV and the signal-to-noise ratio is more than 5 dB, with the noise level being measured at times when there is no electrical stimulation of the cochlea.

16. A method for automated auditory nerve screening by measuring neural responses at the implanted electrodes of a cochlear implant device, comprising:
setting a maximum electrode current for each electrode;
measuring, by a screening device connected to the cochlear implant device, for each electrode, the impedance of the electrode and judging, for each electrode, whether the electrode is a valid electrode or not according to the measured impedance of the electrode;
estimating, by the screening device, the threshold current level required for achieving an ECAP-signal for each valid electrode based on interpolated growth function measurements of the ECAP-signal at at least 4 current levels;
conducting, by the screening device, recovery measurements for some of those electrodes for which an ECAP level of at least 100 μV was measured in the growth function measurements, the recovery measurements comprising ECAP measurements at constant current level as a function of an interpulse interval of the stimulation signal, thereby obtaining a recovery function for each of the recovery-measured electrodes, and obtaining a recovery time constant for each of the recovery-measured electrodes from an exponential fit of the respective recovery function;
conducting, by the screening device, a spread-of-excitation (SOE) measurement for some of those electrodes for which an ECAP level of at least 100 μV was measured in the growth function measurements, wherein the stimulation current is applied subsequently only to one of the electrodes as the probe electrode while an ECAP level is measured subsequently at each of the valid electrodes in order to obtain the spread-of-excitation function; and obtaining, for each electrode used as a probe electrodes a width of electrical stimulation from a fit of the spread-of-excitation function;
wherein a measurement point in all measurements is accepted, by the screening device, as a valid ECAP measurement only if the ECAP signal is at least 50 μV and SNR is more than 5 dB, with the noise level being measured at times when there is no electrical stimulation of the cochlea.

17. The method of claim 16, wherein for each valid electrode the current resulting in an ECAP level of 100 μV is determined by the screening device, and wherein in the recovery measurements and/or in the SOE measurements the applied current for each electrode is at least the determined current resulting in an ECAP level of 100 μV but not more than the set maximum current.

18. A method for automated auditory nerve screening by measuring neural responses at implanted electrodes of a cochlear implant device,
wherein a screening device cooperates with the cochlear implant device in a manner so as to provide the electrodes with stimulation signals suitable for conducting growth function measurements, and wherein the screening device conducts a growth function measurement for estimating the threshold current level required for achieving a valid ECAP-signal at a certain electrode, the growth function measurement comprising the following steps:
a first step with an ECAP measurement at a first current level, which is at least 70% of the maximum allowable current level;
a second step with an ECAP measurement at a second current level which is not more than 20% of the maximum allowable current level, with no valid ECAP-level being expected at the second current level;
a third step with an ECAP measurement at a third current level in-between the first current level and the second current level, wherein, if no valid ECAP-level can be measured at the third current level, the value of the third current level is increased in a stepwise manner until a valid ECAP-level is measured;
a fourth step, wherein an ECAP threshold is estimated from the slope of the valid ECAP levels obtained in the first to third steps, and an ECAP-level is measured at a fourth current level close to, within +/−25% of, the current level corresponding to the estimated ECAP threshold, and wherein, if no valid ECAP-level can be measured at the fourth current level, the value of the fourth current level is increased in a stepwise manner until a valid ECAP-level is measured;
a fifth step with an ECAP measurement at a fifth current level in-between the first current level and the third current level, wherein the fifth current level, if necessary, may be increased in a stepwise manner until a valid ECAP-level is measured;
wherein the ECAP threshold is estimated from the slope of a linear fit of the valid ECAP levels obtained in the previous steps.
wherein a measurement point in all measurements generally is accepted by the screening device as a valid ECAP measurement only if the ECAP signal is at least 50 μV and the signal-to-noise ratio is more than 5 dB, with the noise level being measured at times when there is no electrical stimulation of the cochlea.
